(19) United States
(12) Patent Application Publication
(10) Pub. No.: US 2010/0016682 A1
(43) Pub. Date: Jan. 21, 2010

(54) PATIENT MONITORING SYSTEM AND METHOD

(75) Inventors: Rainer Schluess, Bondorf (DE); Vitor M. Vicente Antunes, Herrenberg (DE)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/519,782

(22) PCT Filed: Dec. 18, 2007
(86) PCT No.: PCT/IB07/55205

§ 371 Date: Jun. 18, 2009

(30) Foreign Application Priority Data
Dec. 21, 2006 (EP) 06126768.8

(51) Int. Cl. A61B 5/00

(52) U.S. Cl. 600/301

(57) ABSTRACT

The present invention relates to a patient monitoring system (1). Further the invention relates to a method for monitoring a patient and to a computer program for a patient monitoring system (1). In order to provide a reliable technique for reducing the number of false alarms in a patient monitoring system a patient monitoring system (1) is suggested, which comprises a first sensor device (2) adapted to acquire (101) a first patient signal (10) corresponding to a first physiological parameter of the patient, a second sensor device (3, 4, 5) adapted to acquire (102, 103, 104) a second patient signal (11, 12, 13) corresponding to a second physiological parameter of the patient, said second patient signal (11, 12, 13) comprising an overlay signal (14, 15, 16) caused by the first physiological parameter of the patient, a processing device (7) adapted to determine (105) from the first patient signal (10) a first value of the first physiological parameter of the patient, to determine (106, 107, 108) from the overlay signal (14, 15, 16) of the second patient signal (11, 12, 13) a second value of the first physiological parameter of the patient, and to analyze (109) the first and second value of the first physiological parameter of the patient, and a control device (8) adapted to control (110) a patient monitor alarm system (9) depending on the result of the analysis.

The present invention relates to a patient monitoring system. Further the invention relates to a method for monitoring a patient and to a computer program for a patient monitoring system.

Patient monitoring systems are commonly used for the observation of the condition of the patient in a clinical environment. Today most of these patient monitors are multi parameter patient monitors, which can display a large number of different physiological parameters.

One of the main tasks of a modern patient monitoring system is to alert the medical staff, i.e. trigger an alarm signal, in case one or more parameter shows an undesired (critical) behavior or in other words, in case the physiological signals of the patient exceed a predefined upper or lower alarm limit. Although signal-processing performance has been enhanced during the last couple of years, there are still a lot of false positive (single) alarms. These are alarms, which are triggered in the patient monitoring system although the status of the patient has not really changed. Such alarms are caused mainly by motion artifacts of the patient, by signal noise, by bad sensor placement or by manual procedures (like flush/sample for the invasive blood pressure parameter) and they cannot easily be completely eliminated by signal processing without either increasing the alarm delay time of a physiological parameter or filtering out ‘true’ alarm conditions. An example for a bad sensor placement is the case of an ECG monitoring, during which the electrodes are used additionally for a respiration impedance measurement. In such a case the electrodes are often placed on the patient's body in a way optimized for ECG signal quality, but not optimized for respiration signal quality, leading to an increased number of false respiration alarms. Since a large number of false alarms in a clinical environment reduces the awareness of the medical personnel to true (relevant) alarms or even leads to the fact that the sourcing parameter alarms are simply turned off, the quality of patient monitoring is reduced by these false alarms.

Modern patient monitoring systems try to reduce the number of false positive alarms by means of filtering the physiological signal and/or by announcing such alarms only if the alarm condition is fulfilled for a predefined period of time. In addition, special algorithms have been developed for the detection of specific cases like e.g. flush/sample detection for invasive blood pressure measurement to filter out false alarm annunciation in a patient monitoring system. However, these mechanisms have been developed for special cases only (see above flush/sample detection algorithms). There is no general technique for use with other physiological parameter or for general use with a large number of physiological parameter. Furthermore the use of these known algorithms might increase the alarm annunciation delay for true positive alarm conditions. In other word, in case of a true alarm the alarm delay time is increased, which in some cases may harm the patient.

Independent studies in clinical environments show that false positive alarms is one of the most common issues that exists with patient monitors in a clinical environment. It is therefore an object of the present invention to provide a reliable technique for reducing the number of false alarms in a patient monitoring system.

This object of the present invention is achieved by a patient monitoring system, comprising a first sensor device adapted to acquire a first patient signal corresponding to a first physiological parameter of the patient, a second sensor device adapted to acquire a second patient signal corresponding to a second physiological parameter of the patient, said second patient signal comprising an overlay signal caused by the first physiological parameter of the patient, a processing device adapted to determine from the first patient signal a first value of the first physiological parameter of the patient, to determine from the overlay signal of the second patient signal a second value of the first physiological parameter of the patient, and to analyze the first and second value of the first physiological parameter of the patient, and a control device adapted to control a patient monitor alarm system depending on the result of the analysis.

The object is also achieved according to the invention by a method of monitoring a patient, the method comprising the steps of acquiring a first patient signal corresponding to a first physiological parameter of the patient, determining from the first patient signal a first value of the first physiological parameter of the patient, acquiring a second patient signal corresponding to a second physiological parameter of the patient, said second patient signal comprising an overlay signal caused by the first physiological parameter of the patient, determining from the overlay signal of the second patient signal a second value of the first physiological parameter of the patient, analyzing the first and second value of the first physiological parameter of the patient, and controlling the release of a monitoring alarm depending on the result of the analyzing step.

The object of the present invention is also achieved by a computer program for a patient monitoring system, wherein the patient monitoring system is adapted to acquire a first patient signal corresponding to a first physiological parameter of the patient, and a second patient signal corresponding to a second physiological parameter of the patient, said second patient signal comprising an overlay signal caused by the first physiological parameter of the patient. The computer program according to the present invention comprises computer instructions to determine from the first patient signal a first value of the first physiological parameter of the patient, computer instructions to determine from the overlay signal of the second patient signal a second value of the first physiological parameter of the patient, computer instructions to analyze the first and second value of the first physiological parameter of the patient, and computer instructions to control a patient monitor alarm system depending on the result of the analysis, when the computer program is executed in a computer.

During monitoring of a first physiological parameter of a patient a primary signal corresponding to a first physiological parameter of the patient is used. Now, a core idea of the invention is to provide a technique for reducing the number of false positive alarms during monitoring of said first physiological parameter of the patient. For this purpose an additional input signal is employed. Said additional input signal corresponds to a second physiological parameter of the patient, and indirectly contains information (second order information) about the first physiological parameter. The second order information are extracted and used in order to evaluate and/or to verify the first order information obtained through the primary input signal. In other words, both the additional input signal and the primary input signal are used for deciding, whether or not a positive alarm shall be issued. Therefore the number of false positive alarms for the first physiological parameter is considerably reduced and the quality of patient monitoring is increased. At the same time the speed, the reliability and the effectiveness of the recognition of true (relevant) patient alarms are improved. As some of the most common false alarms in a patient monitoring system can be suppressed, the medical staff is no longer distracted from normal work or patient care thus improving the efficiency in a hospital environment.

The term “overlay signal” has to be understood as an information signal that is contained within a primary (raw) signal and can be extracted out of the primary signal by means of correlation, filtering, Fast Fourier Transformation (FFT) or any other means. The extracted information signal may be e.g. an amplitude modulated signal, a frequency modulated signal or frequency spectras (frequency range), or a pulse width modulated signal. However, the extracted information signal is primarily used for deriving a different physiological parameter from the primary signal, which corresponds to another physiological parameter. For example, ECG is usually measured for the heart activity or for ST measurement, pleth is primarily measured to derive the pulse rate and the SpO₂ value, invasive blood pressure is measured to derive the blood pressure of a patient and a pulse rate, but all these signals also contain some information about respiration activity of the patient. In these cases the respiration signal can be seen as an amplitude modulated low frequency signal that is contained in the primary signal.

These and other aspects of the invention will be further elaborated on the basis of the following embodiments, which are defined in the dependent claims.

Preferably, the invention can be employed in combination with respiration measurements, in particular with impedance respiration measurements. Impedance respiration measurement is still the commonly used method for non-ventilated patients to measure the respiration activity of the patient. However, motion artifacts, noisy signals or bad sensor placement can cause false positive respiration rate alarms or even Apnea alarms during the monitoring process. Thus, according to a preferred embodiment of the invention, the first physiological parameter of the patient is the patient's (impedance measured) respiration and the overlay signal of the second patient signal is a respiration overlay signal. Although the number of false positive respiration alarms within a patient monitor is very high, this is the first reliable technical solution related to false positive respiration alarms. The present invention can however also be applied, if the patient's respiration is measured in another way, i.e. without impedance measurements, e.g. using the airway CO₂ signal changes between expiration and inspiration or by using a temperature probe that is attached near the patient's nose and temperature changes between inspiration and expiration can be used to derive respiration activity of the patient, but both signals are much less error prone than the impedance measurement.

The respiration overlay signal can be contained in different physiological signals, e.g. in the patient's invasive blood pressure, in the patient's plethysmogram signal or even in the ECG signal of the patient. Accordingly, the second physiological parameter of the patient is the patient's invasive blood pressure (preferably pulmonary arterial pressure PAP or central venous pressure CVP, as these are measured directly in the patient's chest) and/or the pleth signal of the patient and/or the patient's ECG signal.

Blood pressure, ECG signal and pleth signal can be used alternatively or in combination to obtain additional information about the patient's respiration.

In order to use the additional information (e.g. a respiration signal) contained in the second patient signal (e.g. the invasive blood pressure), this information has to be extracted from said signal. For this purpose the second signals are preferably filtered using appropriate signal filter means and/or processed by appropriate signal processing means. For example the respiration signal can be extracted from the blood pressure signal by low pass filtering, or adaptive moving average, by using the envelope of the blood pressure (wave) signal, or by using a Fast Fourier Transformation FFT technique, etc.

Once the additional information have been obtained and analyzed, the patient monitoring system uses this information in order to reduce the number of false alarms with respect to the monitoring of the first physiological parameter. According to a preferred embodiment of the invention an alarm related to the first physiological parameter is suppressed if the second value of the physiological parameter to be monitored is non-critical, even in case of the first value of said physiological parameter being critical.

These and other aspects of the invention will be described in detail hereinafter, by way of example, with reference to the following embodiments and the accompanying drawings; in which:

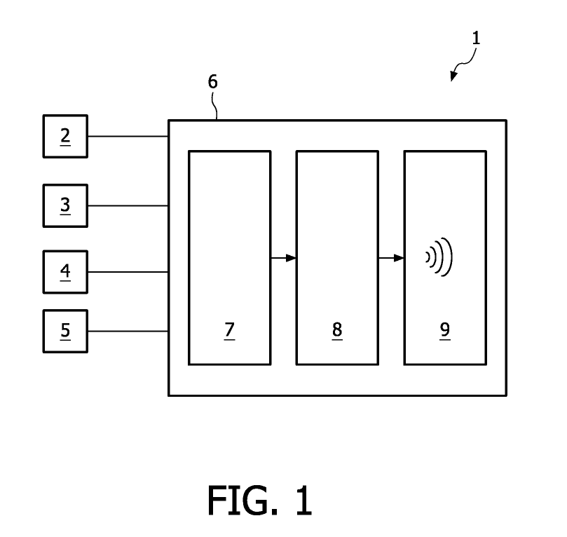

FIG. 1 shows a schematic block diagram of a patient monitoring system according to the invention,

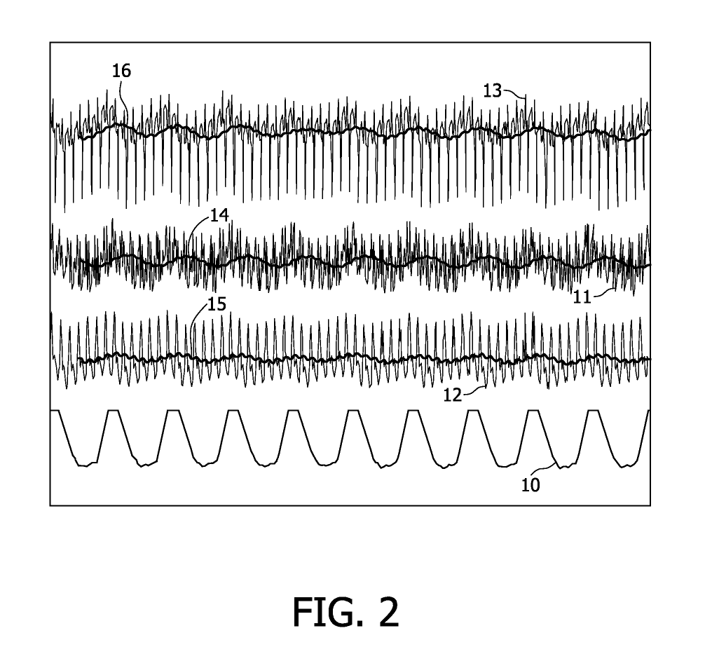

FIG. 2 shows a monitoring chart comprising ECG signal, PAP signal, pleth signal and respiration signal (from top to bottom),

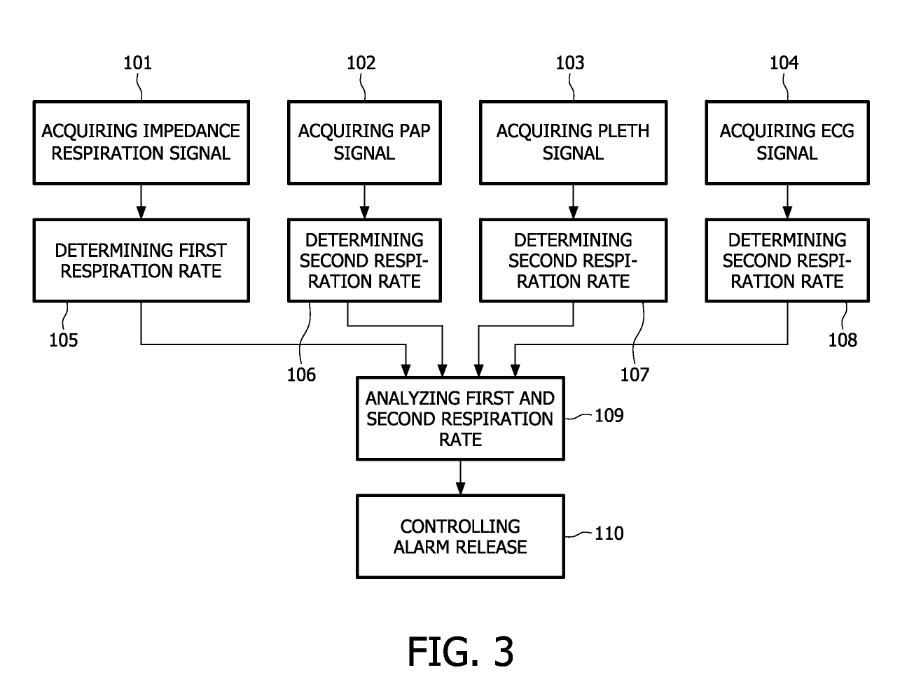

FIG. 3 shows a simplified flowchart of the method according to the invention, and

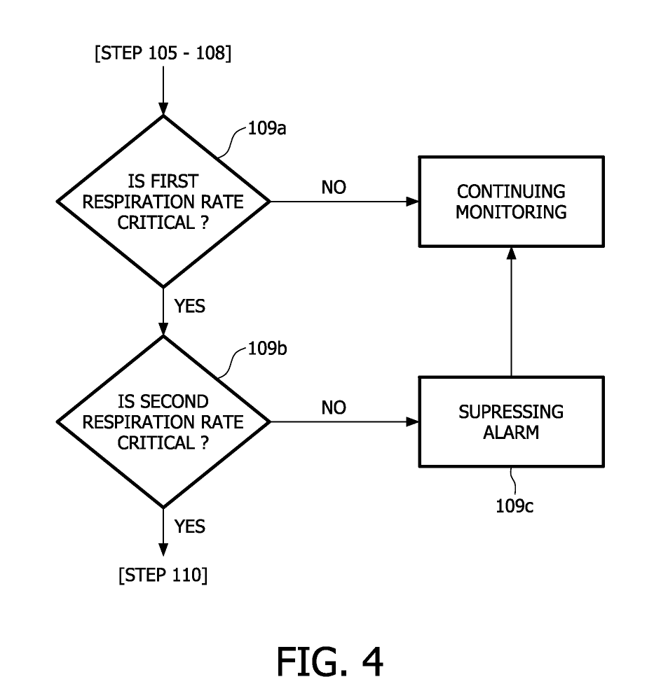

FIG. 4 shows a simplified flowchart of the analysing step of the above method.

A patient monitoring system 1 according to the present invention is illustrated in FIG. 1. With the monitoring system 1 as described, a respiration related false positive Apnea alarm (e.g. caused by motion artefacts or noisy signal) is suppressed by using an invasive blood pressure signal of a patient as alternative parameter.

The system 1 comprises a number of measuring electrodes 2, which are adapted for an impedance respiration measurement in order to obtain a respiration signal 10, corresponding to the respiration of the patient, see FIG. 2. The system 1 further comprises a measuring device 3 for sensing the patient's invasive blood pressure (PAP) 11, and a SpO₂ sensor 4 for acquiring the patient's plethysmogram signal (pleth signal) 12, corresponding to pulsation of the patient's blood at the sensor measurement point, and an ECG sensor 5 for measuring an ECG signal 13. All signals, the PAP signal 11, the pleth signal 12 and the ECG signal 13 comprise a respiration overlay signal 14, 15, 16 caused by respiration of the patient, see FIG. 2. For acquiring patient signals the system 1 may further comprise additional equipment (not shown), like analogue/digital converter, cabling, etc. as known in the art.

The system 1 further comprises a patient monitor 6. The patient monitor 6 comprises a computer, which realizes a processing device 7 and a control device 8. For this purpose a standard multi parameter patient monitor may be used, which is provided with an appropriate software update.

After the (impedance) respiration signal 10 of a patient has been acquired (step 101), and at least one of the related signals such as the PAP signal 11, the pleth signal 12 and the ECG signal 13 have been acquired (steps 102, 103, 104), the processing device 7 determines from the respiration signal 10 a first respiration rate of the patient in step 105. For this purpose the (impedance) respiration signal 10 is filtered and an averaged RRᵢₘₚ and RR_{ECG} numeric is calculated. Due to the filtering and averaging the signal output is delayed.

Since at least one other signal, invasive blood pressure 11, pleth signal 12 or ECG signal 13 of the patient are used to obtain second order information about the patient's respiration, the processing device 7 further determines second respiration rates from the respiration overlay signal 14 of the PAP signal 11 (RR_{PAP}), and/or from the respiration overlay signal 15 of the pleth signal 12 (RR_{Pleth}) and/or from the respiration overlay signal 16 of the ECG signal 13 in steps 106, 107, 108. The respiration overlay signals illustrated in FIG. 2 have been calculated by the processing device 7 using the moving average approach. Alternatively a signal filtering or a Fast Fourier Transformation can be applied to the PAP signal 11 and/or the pleth signal 12 and/or the ECG signal 13 by means of the processing device 7 in order to extract the respiration overlay signals.

The processing device 7 is further adapted to analyze the first and second values of the respiration rate of the patient (step 109). Depending on the results of the analysis an alarm is triggered (step 110) by means of the control device 8, which is adapted to control a patient monitor alarm system 9 depending on the result of the analysis.

With respect to FIG. 4, the analyzing step 109 is described in more detail below. During the analysis process the processing device 7 determines (step 109a), if the first respiration rate is critical, i.e. exceeds a predefined upper or lower alarm limit (or any other threshold). A threshold may for example be defined as a certain time duration, e.g. 20 sec of no respiration activity, that might leads to an Apnea alarm)

In particular, the processing device 7 tries to detect any respiration activity within a predefined time period (e.g. Apnea detection timeout to suppress the ‘false’ Apnea alarms or a different time period for e.g. respiration low or high limit alarms). If the first respiration rate is critical, the processing device 7 announces an alarm to the alarm system 9 of the patient monitor 6, said alarm will however not be released. The announced alarm contains information about the time the critical respiration rate has been determined.

Further the processing device 7 determines (step 109b), whether the second respiration rate obtained from the currently measured (real time) PAP signal 11 or the second respiration rate obtained from the currently measured (real time) pleth signal 12 or the second respiration rate obtained from the currently measured (real time) ECG signal 13 is critical. If a valid respiration overlay signal 14, 15, 16 is detected and the second respiration rate is similar to the first respiration rate (before it went critical). In other words if the determined second respiration rate is evaluated non-critical, the Apnea alarm annunciation is suppressed (step 109c). The suppression period can either be infinite, i.e. as long as a respiration like signal is extracted from the PAP signal 11 or from the pleth signal 12 or from the ECG signal 13, or the suppression could be time limited (e.g. 10 sec) to suppress only artefactual respiration signals. Only if the second respiration rate is critical as well, the announced alarm is released in step 110.

All appliances of the patient monitoring system are adapted to carry out the method according to the present invention. All devices, e. g. the sensor device, the processing device and the control device, are constructed and/or programmed in a way that the procedures for obtaining signals and/or data and for signal and/or data processing run in accordance with the method of the invention.

The monitor with its devices 7, 8, 9 is adapted for performing all tasks of calculating and computing the acquired signals and/or data as well as determining, analyzing and assessing results and controlling. This is achieved according to the invention by means of computer software comprising computer instructions adapted for carrying out the steps of the inventive method, when the software is executed in the patient monitor 6. The patient monitor 6 itself or its devices 7, 8, 9 may for this purpose comprise functional modules or units, which are implemented in form of hardware, software or in form of a combination of both.

The technical effects necessary according to the invention can thus be realized on the basis of the instructions of the computer program in accordance with the invention. Such a computer program can be stored on a carrier such as a CD-ROM or it can be available over the internet or another computer network. Prior to executing the computer program is loaded into the computer by reading the computer program from the carrier, for example by means of a CD-ROM player, or from the internet, and storing it in the memory of the computer. The computer includes inter alia a central processor unit (CPU), a bus system, memory means, e.g. RAM or ROM etc., storage means, e.g. floppy disk or hard disk units etc. and input/output units. Alternatively, the inventive method could be implemented in hardware, e. g. using one or more integrated circuits.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” do not exclude a plurality, and that a single element, such as a computer system or another unit may fulfil the functions of several means recited in the claims. Any reference signs in the claims shall not be construed as limiting the claim concerned.

### REFERENCE NUMERALS

1 patient monitoring system

2 electrodes

3 blood pressure measuring device

4 SpO₂ measuring device

5 ECG measuring device

6 patient monitor

7 processing device

8 control device

9 alarm system

10 respiration signal

11 PAP signal

12 pleth signal

13 ECG signal

14 respiration overlay signal (derived from invasive blood pressure signal)

15 respiration overlay signal (derived from pleth signal)

16 respiration overlay signal (derived from ECG signal)

101-110 method steps

1. A patient monitoring system, comprising
a first sensor device adapted to acquire a first patient signal corresponding to a first physiological parameter of the patient,
a second sensor device adapted to acquire a second patient signal corresponding to a second physiological parameter of the patient, said second patient signal comprising an overlay signal caused by the first physiological parameter of the patient,
a processing device adapted to determine from the first patient signal a first value of the first physiological parameter of the patient, to determine from the overlay signal of the second patient signal a second value of the first physiological parameter of the patient, and to analyze the first and second value of the first physiological parameter of the patient, and
a control device adapted to control a patient monitor alarm system (9) depending on the result of the analysis.

2. The system as claimed in claim 1, wherein the first physiological parameter of the patient is the patient's respiration and the overlay signal of the second patient signal is a respiration overlay signal.

3. The system as claimed in claim 1, wherein the second physiological parameter of the patient is the patient's invasive blood pressure.

4. The system as claimed in claim 1, wherein the second physiological parameter of the patient is the pulsation of the patient's blood.

5. The system as claimed in claim 1, wherein the second physiological parameter of the patient is the electrical voltage in the patient's heart.

6. A method of monitoring a patient, the method comprising the steps of
acquiring a first patient signal corresponding to a first physiological parameter of the patient,
determining from the first patient signal a first value of the first physiological parameter of the patient,
acquiring a second patient signal corresponding to a second physiological parameter of the patient, said second patient signal comprising an overlay signal caused by the first physiological parameter of the patient,
determining from the overlay signal of the second patient signal a second value of the first physiological parameter of the patient,
analyzing the first and second value of the first physiological parameter of the patient, and
controlling the release of a monitoring alarm depending on the result of the analyzing step.

7. The method as claimed in claim 6, wherein in case of the first value being critical an alarm is suppressed, if the second value is non-critical.

8. A computer program for a patient monitoring system, which is adapted to acquire a first patient signal corresponding to a first physiological parameter of the patient, and a second patient signal corresponding to a second physiological parameter of the patient, said second patient signal comprising an overlay signal caused by the first physiological parameter of the patient, the program comprising:
computer instructions to determine from the first patient signal a first value of the first physiological parameter of the patient,
computer instructions to determine from the overlay signal of the second patient signal a second value of the first physiological parameter of the patient,
computer instructions to analyze the first and second value of the first physiological parameter of the patient, and
computer instructions to control the release of a monitoring alarm depending on the result of the analysis,
when the computer program is executed in a computer.
